(19) United States
(12) Patent Application Publication
(10) Pub. No.: US 2019/0125982 A1
(43) Pub. Date: May 2, 2019

(54) RADIAL NEEDLE STORAGE MAGAZINE

(71) Applicant: Becton, Dickinson and Company, Franklin Lakes, NJ (US)

(72) Inventors: Sudarsan Srinivasan, North Brunswick, NJ (US); David Schiff, Highland Park, NJ (US); Stephan Lawson, Malvern, PA (US)

(73) Assignee: Becton, Dickinson and Company, Franklin Lakes, NJ (US)

(21) Appl. No.: 16/096,165

(22) PCT Filed: Mar. 31, 2017
(86) PCT No.: PCT/US17/25429

§ 371 Date: Oct. 24, 2018

Related U.S. Application Data
(60) Provisional application No. 62/328,682, filed on Apr. 28, 2016.

(51) Int. Cl. A61M 5/32; A61M 5/00; A61M 5/34

(52) U.S. Cl. CPC A61M 5/3297; A61M 5/002; A61M 2005/3125; A61M 5/345; A61M 2005/004; A61M 5/3293

(57) ABSTRACT

A magazine (16) that stores a plurality of needles (56) configured to engage a medication pen (2) for medication delivery, the magazine (16) comprising a magazine housing (32, 70) enclosing a removable array (26) that is rotatable in the magazine housing (32, 70), the removable array (26) supporting a plurality of hub chambers (22) each enclosing one of a plurality of needle hubs (50), the plurality of hub chambers (22) being radially disposed, a connector (28) in each of the plurality of hub chambers (22), each connector (28) engages one of the plurality of needle hubs (50), and a plurality of closures (30) each sealing the plurality of needle hubs (50), wherein a selected closure of the plurality of closures (30) is pierced by the medication pen (2) to engage a selected needle hub (52) of the plurality of needle hubs (50), the selected needle hub (52) disengages from the hub chamber (24), and the selected needle hub (52) is removed from the magazine housing (32, 70) to prepare the medication pen (2) for medication delivery.

This application claims the benefit under 35 U.S.C. § 119(e) of U.S. Provisional Patent Application Ser. 62/328,682 filed on Apr. 28, 2016, which is hereby incorporated by reference in its entirety.

### FIELD

Various exemplary embodiments of the invention relate to needle storage for medication pen needles.

### BACKGROUND

Medication pens are typically used to inject medication into a patient. A person who must periodically self-inject doses of medication will typically carry a medication pen and several single-use pen needles. A medication pen is designed for safety and sterility. However, inefficiencies and inconveniences arise.

### SUMMARY OF THE INVENTION

It is an aspect of the present invention to provide a radial magazine that stores a plurality of needles configured to engage a medication pen for medication delivery. Such a radial magazine provides advantages in minimizing the size of pen needle packaging, minimizing the volume of plastic used to manufacture components, and grouping together a number of pen needles in a radial arrangement to make them easy to use and carry. These advantages are provided in a radial magazine having a size and shape similar to a poker chip.

Having a radial magazine of needles available for medication delivery reduces needle reuse. Needle reuse is undesired for at least the following reasons. The needle dulls after a single use and so subsequent use may cause pain to the patient. Multiple needle use can also reduce the strength of the needle tip which may cause a potential fracture. Also, needle reuse increases sanitary concerns and health risks to the patient.

The radial needle assembly of the present invention advantageously reduces reuse for at least the following reasons. Although patients may desire to financially benefit from using a needle multiple times, the needle assembly is configured to prevent each of the plurality of needles from being used more than once. Convenience is another reason patients reuse needles. Patients may also be concerned about not having another needle available for use or not having access to supplies. However, the needle assembly conveniently provides multiple needles so that an unused needle is more readily available.

The foregoing and/or other aspects of the present invention can be achieved by providing a magazine that stores a plurality of needles configured to engage a medication pen for medication delivery, the magazine comprising a magazine housing enclosing a removable array that is rotatable in the magazine housing, the removable array supporting a plurality of hub chambers each enclosing one of a plurality of needle hubs, the plurality of hub chambers being radially disposed, a connector in each of the plurality of hub chambers, each connector engages one of the plurality of needle hubs, and a plurality of closures each sealing the plurality of needle hubs, wherein a selected closure of the plurality of closures is pierced by the medication pen to engage a selected needle hub of the plurality of needle hubs, the selected needle hub disengages from the hub chamber, and the selected needle hub is removed from the magazine housing to prepare the medication pen for medication delivery.

Preferably, a centerline of each of the plurality of hub chambers substantially intersects at a centerline of the magazine housing. The magazine housing is a substantially circular shape and includes a cover and a base. The magazine further includes a fastener to secure the cover to the base.

Preferably, the magazine housing includes a window to display a number identifying the needle hub in use. The magazine housing includes an opening to provide access to the selected needle hub, the opening provides access to only one needle hub at a time and the opening is aligned to the selected needle hub.

Preferably, the magazine includes a ratcheting slider for indexing the plurality of hub chambers in the magazine housing. The ratcheting slider includes a ratchet shield that obstructs an opening in the magazine housing during indexing. The ratchet shield provides access to the opening before and after indexing and obstructs the opening during indexing. The ratcheting slider moves along an outer circumference of the magazine housing.

Preferably, the magazine includes a locking member to prevent reverse rotation of the removable array. The magazine housing and the removable array each includes a rib that engage to prevent the magazine from exposing the plurality of needle hubs that are used.

Preferably, the removable array is replaced when the plurality of needle hubs is used. Each of the hub chambers is identified by a hub chamber number. One of the plurality of hub chamber numbers aligns with a window in the magazine housing for display.

Preferably, the magazine housing further includes a guide track to engage and disengage a ratcheting slider. The magazine housing also includes a ramp, and the ramp is adjacent to the guide track to engage and disengage the ratcheting slider from the removable array.

The foregoing and/or other aspects of the present invention can further be achieved by providing a method of using a plurality of needles in a magazine, the plurality of needles is configured to engage a medication pen for medication delivery, the method comprising indexing a selected needle hub of a plurality of needle hubs to align with an opening in a magazine housing, piercing a selected closure of a plurality of closures that seals the selected needle hub with the medication pen, engaging the medication pen to the selected needle hub in the magazine housing, disengaging the selected needle hub from the magazine housing, and removing the selected needle hub from the magazine housing to prepare the medication pen for medication delivery.

Additional and/or other aspects and advantages of the present invention will be set forth in the description that follows, or will be apparent from the description, or may be learned by practice of the invention.

### BRIEF DESCRIPTION OF THE DRAWINGS

The above aspects and features of the present invention will be more apparent from the description for the exemplary embodiments of the present invention taken with reference to the accompanying drawings, in which:

FIG. 1 illustrates a top exploded view of an exemplary magazine;

FIG. 2 illustrates a front, right exploded view of the magazine;

FIG. 3 illustrates a right perspective view of the magazine;

FIG. 4 illustrates a left perspective cross-sectional view of the magazine;

FIG. 5 illustrates a rear, right exploded view of the magazine;

FIG. 6 illustrates a top view of a base;

FIG. 7 illustrates a top perspective view of the base with a ratcheting slider in a first position;

FIG. 8 illustrates a top sectional view of the base and the ratcheting slider in the first position;

FIG. 9 illustrates a top view of the base with the ratcheting slider moving away from the first position;

FIG. 10 illustrates a top view of the base with the ratcheting slider in a second position;

FIG. 11 illustrates a top view of the base with the ratcheting slider moving away from the second position and toward the first position;

FIG. 12 illustrates a top view of the base with the ratcheting slider in the first position;

FIG. 13 illustrates a top perspective view of a magazine array;

FIG. 14 illustrates a left perspective view of a magazine assembly including the magazine and a medication pen;

FIG. 15 illustrates the right perspective view of an adapter connected to the medication pen;

FIG. 16 illustrates a partial cross sectional view of the medication pen of FIG. 15 beginning to connect to a needle hub in the magazine;

FIG. 17 illustrates a partial cross sectional view of the medication pen of FIG. 15 fully engaged to the needle hub in the magazine;

FIG. 18 illustrates a left perspective view of the magazine assembly where the medication pen engaged to the needle hub is removed from the magazine;

FIG. 19 illustrates a right perspective view of the magazine where the medication pen returns the needle hub to the magazine;

FIG. 20 illustrates a right perspective view of the needle hub in the magazine in an unlocked position;

FIG. 21 illustrates a right perspective view of the needle hub in the magazine in a locked position; and

FIG. 22 illustrates a left perspective view of the needle hub.

### DETAILED DESCRIPTION OF EXEMPLARY EMBODIMENTS

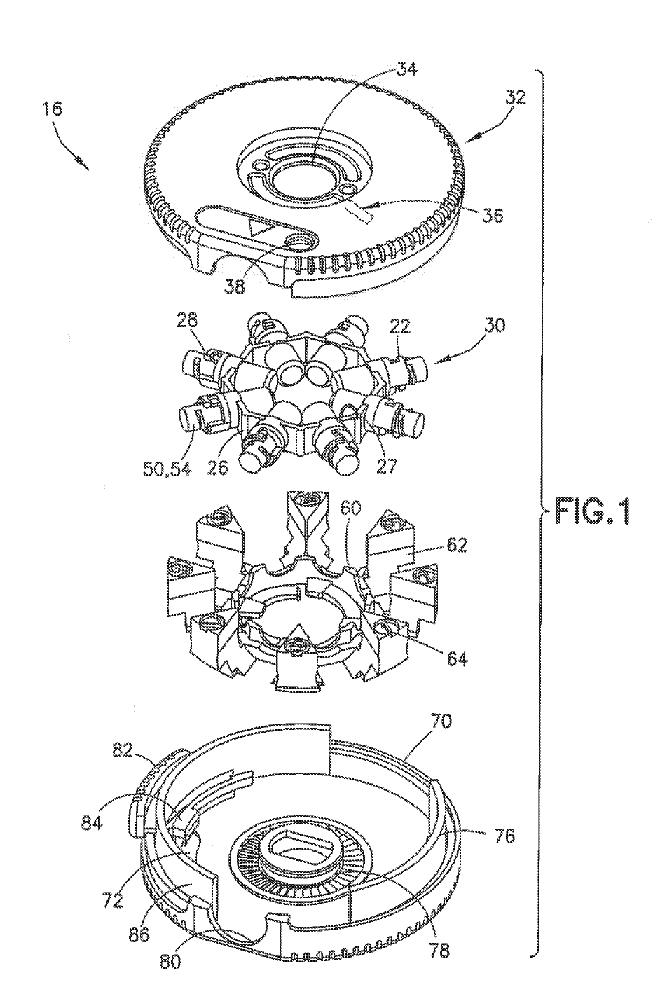

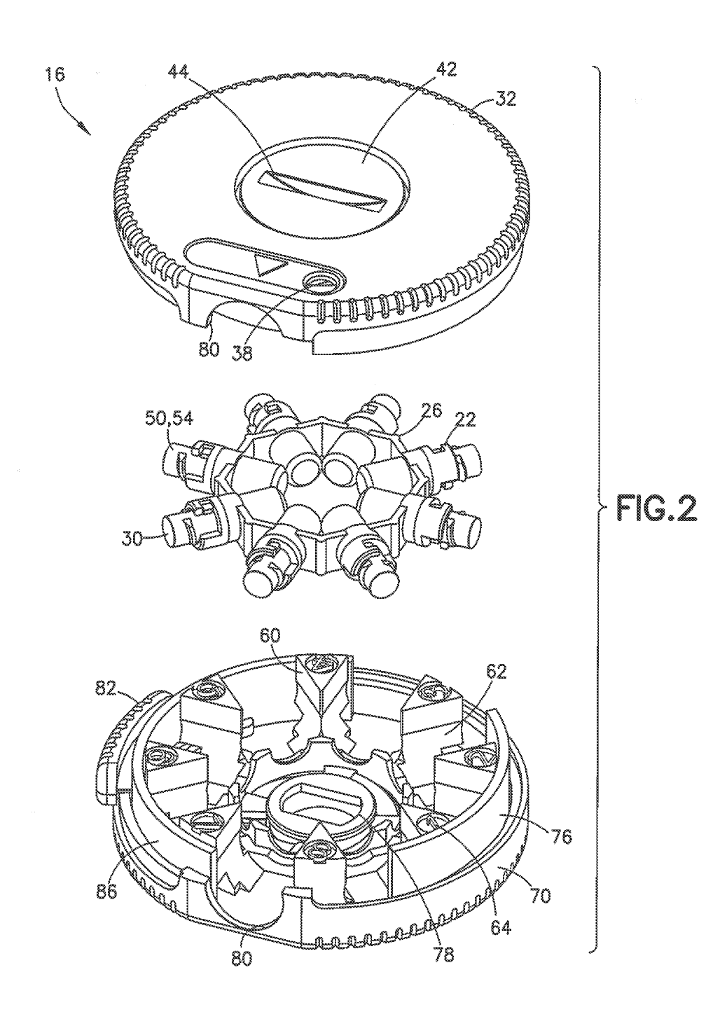

According to one embodiment, FIG. 1 illustrates a radial magazine 16 that stores a plurality of needles or cannulas used by a medication pen for medication delivery. The radial magazine 16 preferably includes a magazine housing having a top cover 32 and a base 70. As illustrated in FIGS. 2-5, the top cover 32 and the base 70 are secured to each other by a fastener 42. The magazine housing 32, 70 is in a circular shape of a poker chip and encloses each of the plurality of needles or cannulas.

The top cover 32 includes a locking hole 34, a rib 36, a window 38 and a needle hub opening 80. The locking hole 34 is a hole that extends through the top cover 32 and is configured to engage the fastener 42 to secure the top cover 32 to the base 70. The locking hole 34 is surrounded by a recessed surface at an outer surface of the top cover 32 for proper engagement to the fastener 42.

As illustrated in FIG. 1, an inner surface of the top cover 32 includes a rib 36. The rib 36 acts as a stopping surface to prevent further indexing after all the needle hubs 50 are used. Indexing is prevented when the rib 36 in the top cover 32 contacts a magazine rib 27 in the radial magazine 16. At this point, indexing is completed and the needle hubs 50 in the radial magazine 16 are all used. The radial magazine 16 can then be discarded and replaced. This safety lock feature is described in further detail below.

The top cover 32 also includes a window 38. The window 38 is a transparent member on the outer surface of the top cover 32 that displays a hub chamber number or counter 64 identifying which needle hub 50 is being used. Specifically, the window 38 aligns with the hub chamber number 64 on an inner plate 60 as the inner plate 60 rotates during operation. The displayed hub chamber number 64 corresponds to the hub chamber 22 disposed in the inner plate 60 that is aligned to the needle hub opening 80. Operation of the window 38 is described in further detail below.

The needle hub opening 80 is a semicircular opening at an outer circumferential surface of the top cover 32. The needle hub opening 80 is also disposed at an outer circumferential surface of the base 70. The two portions of the needle hub opening 80 cooperate together when the top cover 32 is connected to the base 70 to create a circular opening or hole. The needle hub opening 80 is adjacent to the window 38 and provides access to one of the plurality of needle hubs 50 for use.

As illustrated in FIG. 13, the magazine housing 32, 70 includes a removable radial magazine array 26 of needle hubs 50. The radial magazine array 26 includes a plurality of hub chambers 22 each including a connector 28 and a selected hub chamber 24. The radial magazine array 26 radially spaces the plurality of hub chambers 22 in a substantially circular orientation. Specifically, a centerline of each of the plurality of hub chambers 22 substantially intersects at a centerline of the magazine housing 32, 70. The opening of the hub chambers 22 protrude outwardly with respect to a central axis of the radial magazine array 26.

The radial magazine array 26 carries one of a plurality of needle hubs 50 in each of the plurality of hub chambers 22. Preferably, the radial magazine array 26 includes eight hub chambers 22, although more or fewer are contemplated. Such a configuration advantageously provides a small, compact and optimized arrangement of the plurality of needle hubs 50 and allows the radial magazine 16 to be made from a minimal amount of material. Additionally, the plurality of needle hubs 50 is individually disposed in each of the plurality of hub chambers 22 to advantageously provide a separate enclosure for each needle hub 50.

Each of the plurality of hub chambers 22 includes the connector 28 which secures each of the plurality of needle hubs 50. The connector 28 is preferably a quarter-turn bayonet connection, although a push-pull detent connector can also be used. Further details of the connector 28 and its operation are described below.

The radial magazine array 26 further includes a magazine rib 27. As illustrated in FIGS. 1 and 13, the magazine rib 27 is disposed adjacent to the first needle hub 50 indicated by the hub chamber number 64 being 1. The magazine rib 27 is also disposed at a top surface of the radial magazine array 26. As the radial magazine array 26 rotates in a counterclockwise direction during operation, the magazine rib 27 also rotates. When the last hub chamber 22 indicated by the hub chamber number 64 being 8 is used, the magazine rib 27 contacts the rib 36 in the top cover 32 to prevent further rotation. Thus, the radial magazine 16 is not able to be further indexed. At this point, the user understands that the radial magazine array 26 needs to be replaced.

A distal end of each of the plurality of needle hubs 50 is covered by a seal or closure in the form of a peel tab 30. The peel tabs 30 are preferably foil tabs, specifically a composite layer of foil. Alternately, the peel tabs 30 are made of a polymer layer or a thermoplastic material.

Each peel tab 30 individually seals and provides a sterile environment for each of the plurality of needle hubs 50 disposed in the plurality of hub chambers 22. Such a configuration advantageously creates independent access to each of the plurality of needle hubs 50. As a result, one of the plurality of needle hubs 50 can be used without altering the sterile environment of the remaining needle hubs 50.

One of the plurality of peel tabs 30 is removed or pierced by a medication pen to identify a selected hub chamber 24 of the plurality of hub chambers 22. The selected hub chamber 24 includes a selected needle hub 52 ready for use.

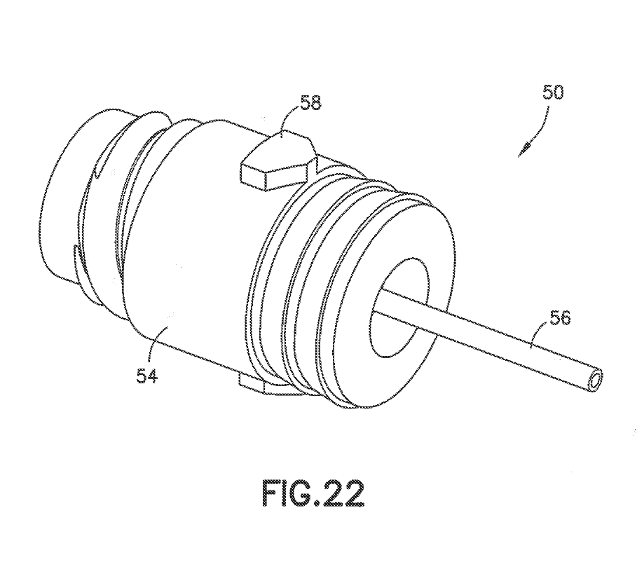

FIG. 22 illustrates, according to one embodiment, one of the plurality of needle hubs 50. The plurality of needles hubs 50 each includes a hub body 54, a hub cannula or hollow needle 56, and a radial lug 58. A proximal and distal end of the hub body 54 preferably includes threads or the push-pull detent. The proximal end of the hub body 54 is configured to be covered by a peel tab 30 and attached to an adapter 4. The distal end of the hub body 54 is configured to be attached to the connector 28 in the radial magazine array 26.

The hub cannula 56 is fixed to the hub body 54 and extends from the distal end of the hub body 54. Although not illustrated, the distal end of the hub cannula 56 includes a sharpened bevel cut that is configured to penetrate tissue. The hub cannula 56 provides a means to deliver medicament to the patient. Specifically, when one of the plurality of needle hubs 50 is connected to the medication pen, fluid communication is established. Accordingly, medicament travels to the needle hub 50 and exits through the hub cannula 56.

The radial lug 58 is disposed adjacent to the threads or the push-pull detent at the distal end of the hub body 54. The radial lug 58 acts as a retention means to the connector 28 in the radial magazine array 26. Two radial lugs 58 are preferably disposed on the hub body 54 approximately 180° apart.

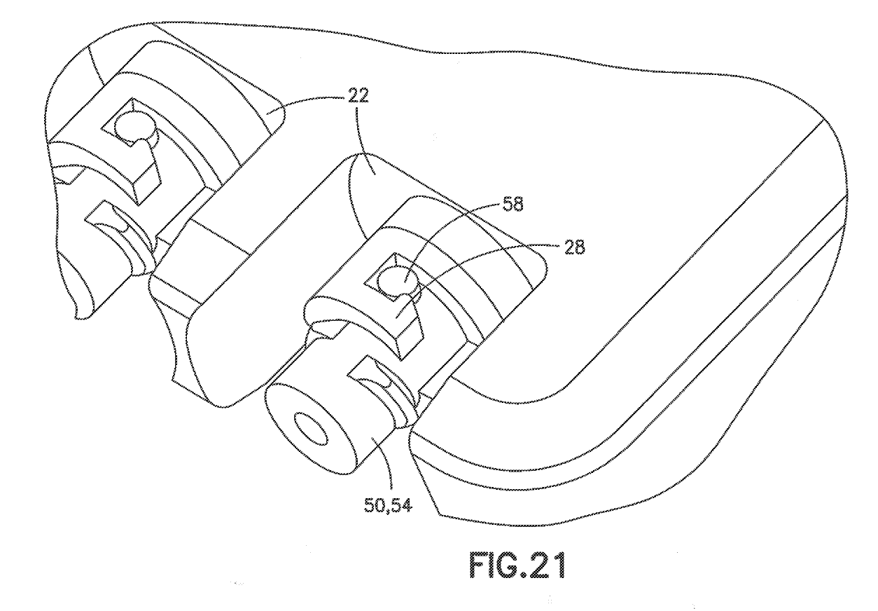

As illustrated in FIG. 20, when the used needle hub 50 is returned to the radial magazine array 26, the used needle hub 50 engages the quarter turn connector 28 via the radial lug 58. The used needle hub 50 is then rotated approximately 90°, as illustrated in FIG. 21, to engage the quarter turn connector 28 of the radial magazine array 26. Thus, the quarter turn connector 28 is secured to the needle hub 52 via the radial lug 58.

According to one embodiment, the radial magazine array 26 is disposed in an inner plate 60. Preferably, as illustrated in FIGS. 1 and 2, the inner plate 60 includes eight hub carrying cavities 62 corresponding to the plurality of hub chambers 22 in the radial magazine array 26. More or fewer hub carrying cavities 62 is contemplated based on the number of corresponding hub chambers 22 in the radial magazine array 26. The hub carrying cavities 62 are a plurality of recessed surfaces that support and partially enclose the plurality of hub chambers 22 of the radial magazine array 26.

The hub chamber number or counter 64 is assigned to each of the hub carrying cavities 62 which sequentially identifies each of the needle hubs 50. The hub chamber number 64 is an extruded surface that aligns with the window 38 of the top cover 32 to identify the selected needle hub 52 being used during operation of the radial magazine 16. This alignment takes place when the radial magazine 16 is not being indexed. The hub carrying cavities 62 are sequentially number in a counterclockwise direction so that when the radial magazine array 26 is rotated in the counterclockwise direction during operation, the hub chamber number 64 increases incrementally from 1 to 8 while being displayed through the window 38 in the top cover 32.

The inner plate 60 is disposed in the base 70 and cooperates with a ratcheting slider 82 to rotate during operation. Further details of the components and the relationship between the inner plate 60, the base 70 and the ratcheting slider 82 are described below,

The base 70, according to one embodiment, is substantially circular and encloses a bottom portion of the radial magazine 16. As described above, the base 70 cooperates with the top cover 32 to fully enclose all the components of the radial magazine 16. The base 70 includes a guide track 72, a ramp 74, a locking member 76, a mounting slot 78 and the needle hub opening 80.

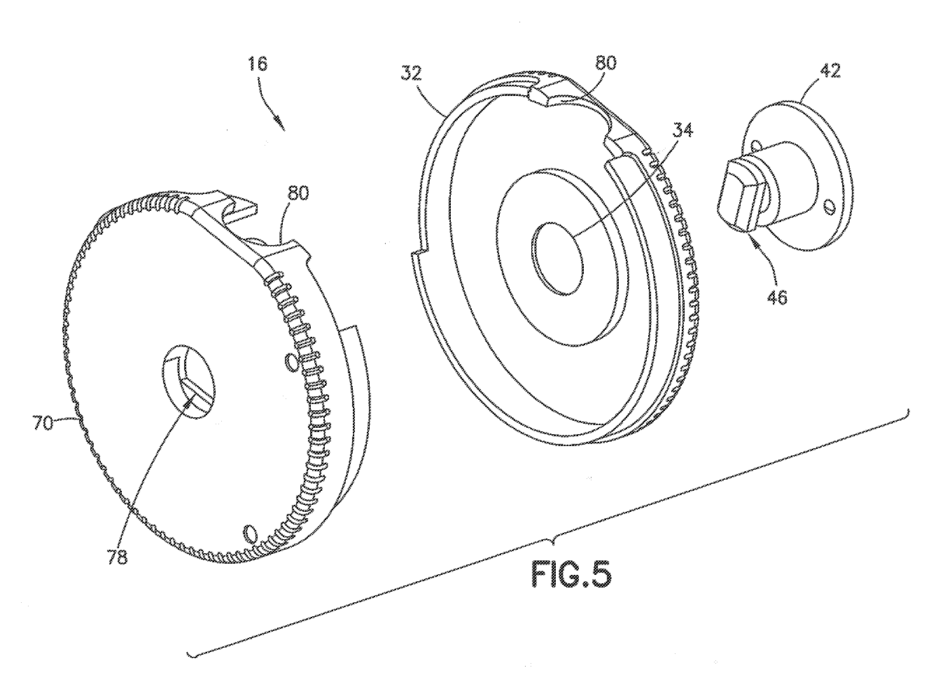

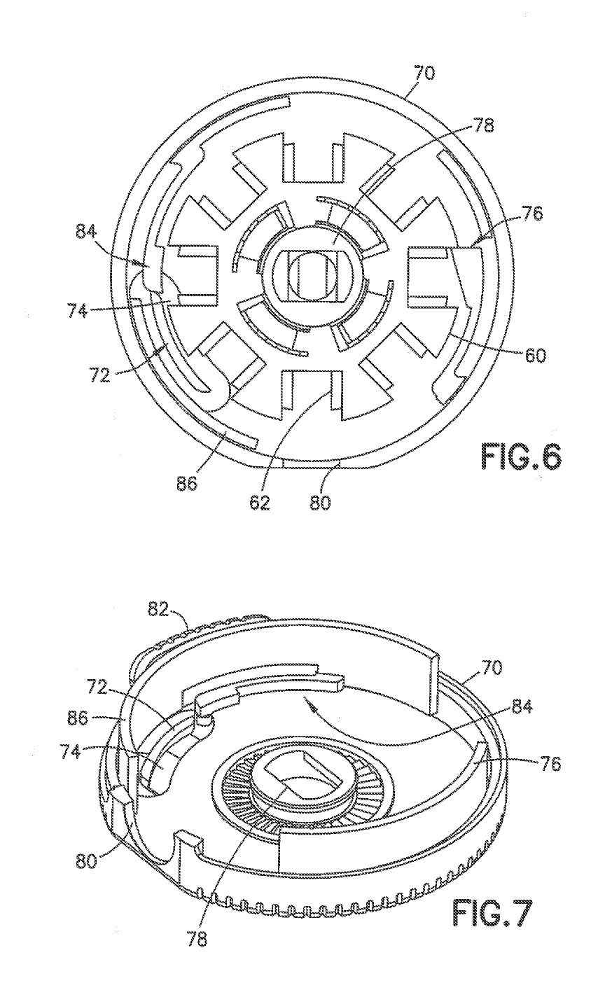

FIGS. 6-12 illustrate the guide track 72 in the base 70. The guide track 72 is a curved protruding surface at a bottom inner surface of the base 70. The guide track directs the circumferential movement of the ratcheting slider 82 between first and second positions.

The base 70 further includes the ramp 74 that surrounds the guide track 72. The ramp 74 is an inclined surface surrounding the guide track 72 that increases in height along a counterclockwise direction. Specifically, the vertical (or axial) distance between a top surface of the guide track 72 and a top surface of the ramp 74 is greatest at the clockwise most portion of the guide track 72 and least at the counterclockwise most portion of the guide track 72. The ramp 74 cooperates with the guide track 72 to direct movement of the ratcheting slider 82. Further information regarding the operation of the ratcheting slider 82 is described below.

The base 70 also includes a locking member 76. The locking member 76 includes a flange portion and a stopping portion. The flange portion is connected at an outer circumference of the base 70 at one end and is connected to the stopping portion at another end. The flange portion allows the stopping portion to move elastically between various positions in a flexible manner.

The stopping portion is an inclined surface that engages the hub carrying cavities 62 of the inner plate 60. The stopping portion allows the inner plate 60 to rotate in a counterclockwise direction during indexing of the radial magazine 16. Specifically, when the inner plate 60 rotates, the stopping portion slides along its inclined surface to disengage one hub carrying cavity 62 and ultimately engage the next adjacent hub carrying cavity 62. The stopping portion prevents the inner plate 60 from rotating in a clockwise direction because the stopping portion is engaged to the hub carrying cavity 62 and creates interference when moved in that direction. In other words, the stopping portion does not have an inclined surface engaging the hub carrying cavities 62 in the clockwise direction.

The mounting slot 78 is a slot that extends through the base 70 and is used to secure the top cover 32 to the base 70 via the fastener 42. Further details regarding the fastener 42 are described below.

As similarly described above, the needle hub opening 80 in the base 70 is a semicircular opening at an outer circumferential surface of the base 70. The needle hub opening 80 cooperates with the outer circumferential surface of the top cover 32 to create a hole providing access to the plurality of needle hubs 50.

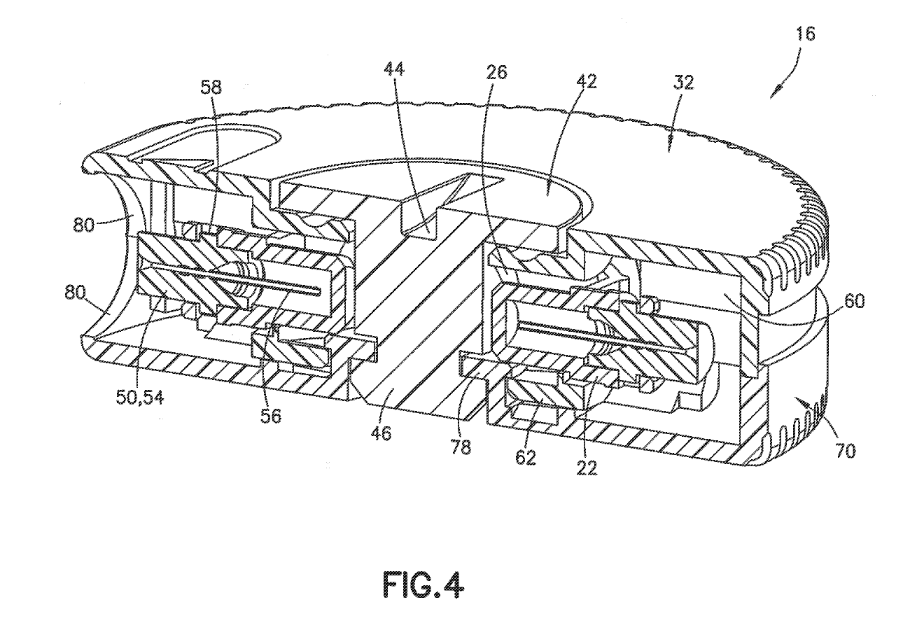

According to one embodiment, as illustrated in FIGS. 4 and 5, the fastener 42 includes a slot 44 and a flat 46. The slot 44 is disposed at a top external surface of the fastener 42 and allows a user to turn the fastener 42. For example, a coin is placed in the slot 44 so that the user can rotate the coin, thus rotating the fastener 42.

The distal end of the fastener 42 includes the flat 46. The flat 46 is configured to enter into the mounting slot 78 of the base 70 and is rotated approximately 90° to lock the flat 46 with respect to the base 70. FIG. 4 illustrates the flat 46 of the fastener 42 locking the top cover 32 to the base 70.

The fastener 42 is rotated approximately 90 to align the flat 46 with the mounting slot 78 of the base 70 to unlock the magazine housing 32, 70. Specifically, the flat 46 is smaller than the mounting slot 78 so that when these components are aligned, the fastener 42 is removed from the base 70. FIG. 4 illustrates the fastener 42 engaged with the top cover 32 and the base 70 to secure the magazine housing 32, 70 together. FIG. 5 illustrates fastener 42 disengaged from the top cover 32 and the base 70.

The radial magazine 16, according to one embodiment, further includes the ratcheting slider 82. The ratcheting slider 82, as illustrated in FIGS. 1-3 and 6-12, includes a driving member 84 and a ratchet shield 86. A portion of the ratcheting slider 82 is disposed on an outer surface of the magazine housing 32, 70 for manipulation by the user. Specifically, the ratcheting slider 82 is moved by the user to index the plurality of needle hubs 50 in the radial magazine 16.

The driving member 84 is a flanged member disposed inside the magazine housing 32, 70 that elastically flexes during operation. The ratchet shield 86 rotates during operation of the ratcheting slider 82 to open and close the needle hub opening 80. Specifically, when the radial magazine 16 is indexing, the ratchet shield 86 covers the needle hub opening 80. When the radial magazine 16 is not indexing, the needle hub opening 80 is open to provide access to the selected needle hub 52 in the selected hub chamber 24. The needle hub opening 80 is sized to provide access to only one needle hub 50 in the selected hub chamber 24 at a time. The operation of the ratcheting slider 82 and the locking member 76 is described as follows.

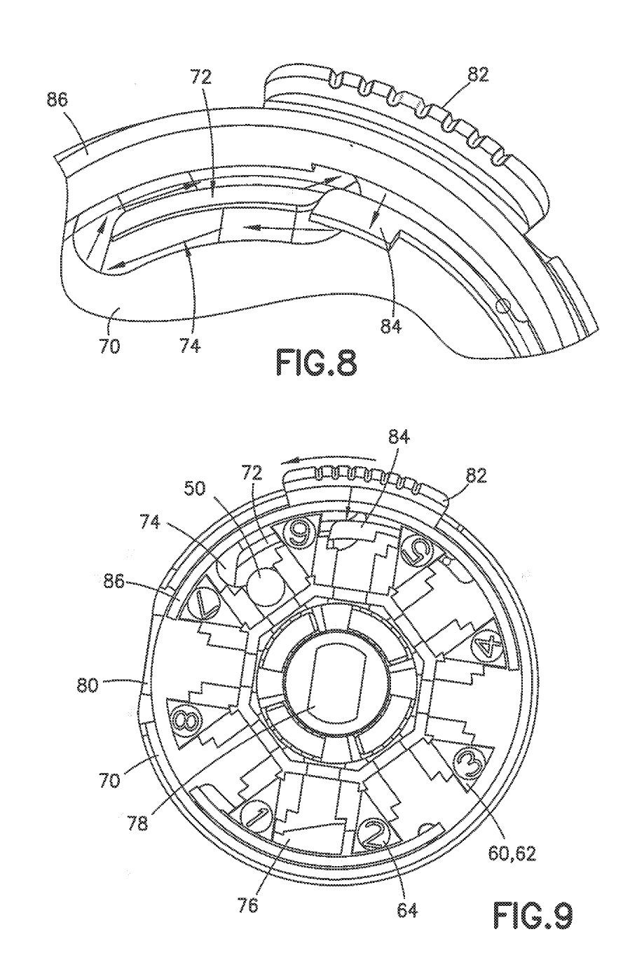

FIGS. 6-8 illustrate the driving member 84 of the ratcheting slider 82 in a first position. The driving member 84 is in its natural state and disengaged from the guide track 72 of the base 70. Additionally, the driving member 84 is disengaged from the hub carrying cavities 62 of the inner plate 60. Finally, the ratchet shield 86 allows access to the needle hub opening 80. FIG. 8 illustrates the travel path of the driving member 84 during operation.

In the first position, the locking member 76 is engaged to one of the hub carrying cavities 62 of the inner plate 60. Specifically, the stopping portion of the locking member 76 enters into the hub carrying cavity 62. In the first position, the locking member 76 prevents the inner plate 60 from rotating in the clockwise direction.

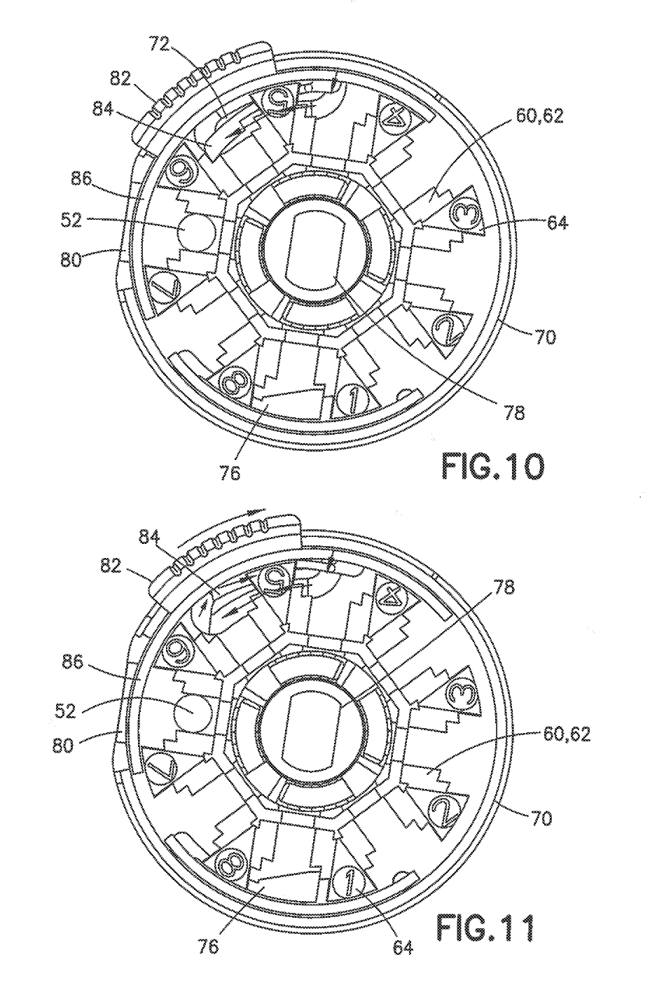

FIG. 9 illustrates the ratcheting slider 82 traveling toward a second position and FIG. 10 illustrates the ratcheting slider 82 in the second position. As the ratcheting slider 82 travels to a second position, the driving member 84 flexes inwardly and is elastically compressed in a radial direction by the guide track 72 to engage the hub carrying cavity 62 of the inner plate 60. Specifically, the driving member 84 is engaged to the inner plate 60 by being disposed in the hub carrying cavity 62. Accordingly, the inner plate 60 is rotated when the ratcheting slider 82 moves along the guide track 72.

As the inner plate 60 rotates in the counterclockwise direction, the locking member 76 moves from one of the hub carrying cavities 62 to an adjacent hub carrying cavity 62. This is because the inclined surface in the stopping portion of the locking member 76 allows for counterclockwise rotation to take place. That is, the stopping portion allows for rotation with respect to the hub carrying cavity 62 at one side of the cavity but not at the other side of the cavity.

FIG. 9 shows the ratcheting slider 82 in the first position and FIG. 10 shows the ratcheting slider 82 in the second position. These Figures also show how the inner plate 60 is indexed so that an adjacent hub chamber 22 is aligned to the needle hub opening 80. At the same time, the ratchet shield 86 moves to prohibit access to the needle hub opening 80. Such a configuration advantageously prevents tampering with the radial magazine 16 during indexing.

The locking member 76 is also indexed or rotated to the adjacent hub carrying cavity 62. Specifically, FIG. 9 illustrates the stopping portion of the locking member 76 between hub chamber numbers 1 and 2 in the first position of the ratcheting slider 82. On the other hand, FIG. 10 illustrates the stopping portion of the locking member 76 between hub chambers numbers 1 and 8 in the second position of the ratcheting slider 82. Similar to the first position, in the second position the ratcheting slider is also locked to prevent reverse movement or reverse indexing of the inner plate 60 in the clockwise direction. Such a configuration advantageously prevents the user from reusing needle hubs 50.

As illustrated in FIG. 8, while the ratcheting slider 82 moves from the first position to the second position in the counterclockwise direction, the driving member 84 moves from one side to another side of the guide track 72 along the ramp 74. The ramp 74 increases in height causing the driving member 84 to gradually disengage from the guide track 72 by the time the driving member 84 reaches the second position.

When the ratcheting slider 82 reaches the second position, the driving member 84 disengages from the guide track 72 and is no longer radially compressed inwardly. Instead, as illustrated in FIG. 10, the driving member 84 rests on top of the guide track 72. The elastic compression of the driving member 84 allows it to return to its original radial position. As a result, the driving member 84 disengages the hub carrying cavity 62 of the inner plate 60.

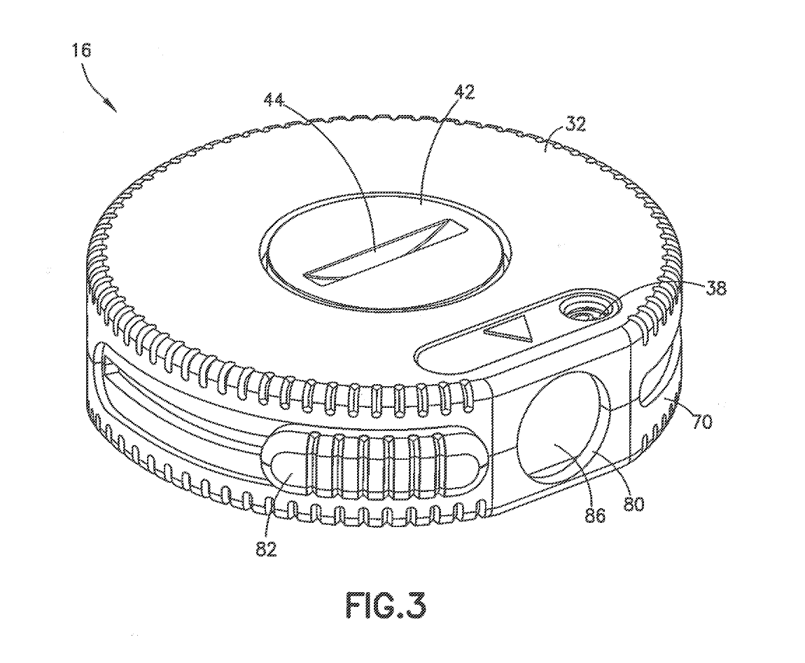

In the second position, as illustrated in FIG. 3, the ratchet shield 86 also moves to cover the needle hub opening 80. Such a configuration advantageously prevents the user from tampering with the radial magazine 16 during indexing of the needle hub 50.

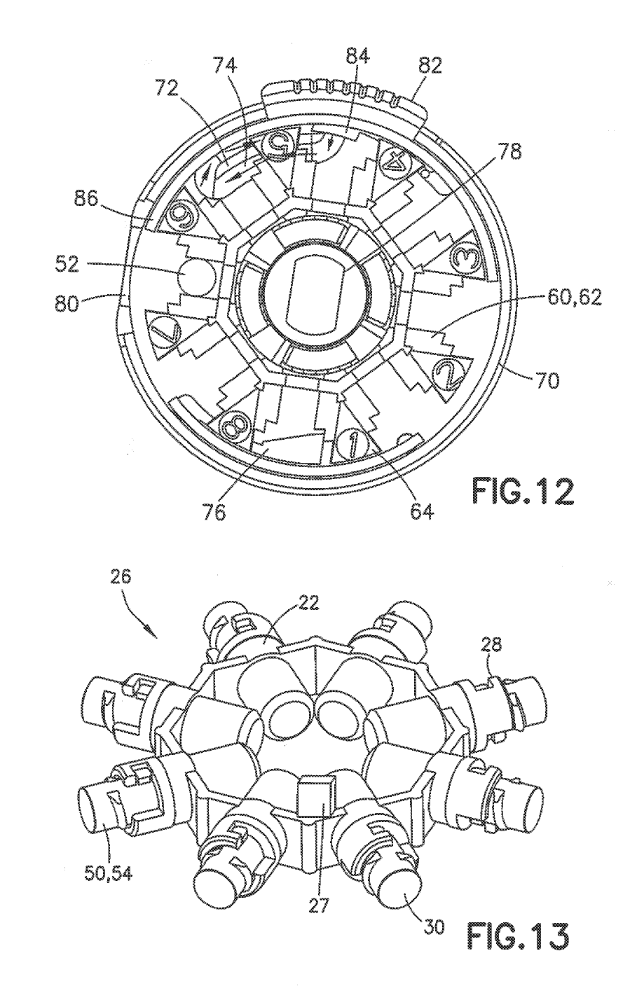

FIG. 11 illustrates the ratcheting slider 82 moving from the second position to the first position and FIG. 12 illustrates the ratcheting slider 82 returning to the first position. As the ratcheting slider 82 moves back to the first position, the driving member 84 slides over the guide track 72. During this motion, since the driving member 84 is disengaged from the hub carrying cavities 62 of the inner plate 60, the inner plate 60 does not rotate when the ratcheting slider 82 returns to the first position. Also, the ratchet shield 86 moves to provide access to the needle hub opening 80. Finally, the locking member 76 stays in the hub carrying cavity 62 between the hub chamber numbers 8 and 1, and prevents the inner plate 60 from rotating in the clockwise direction.

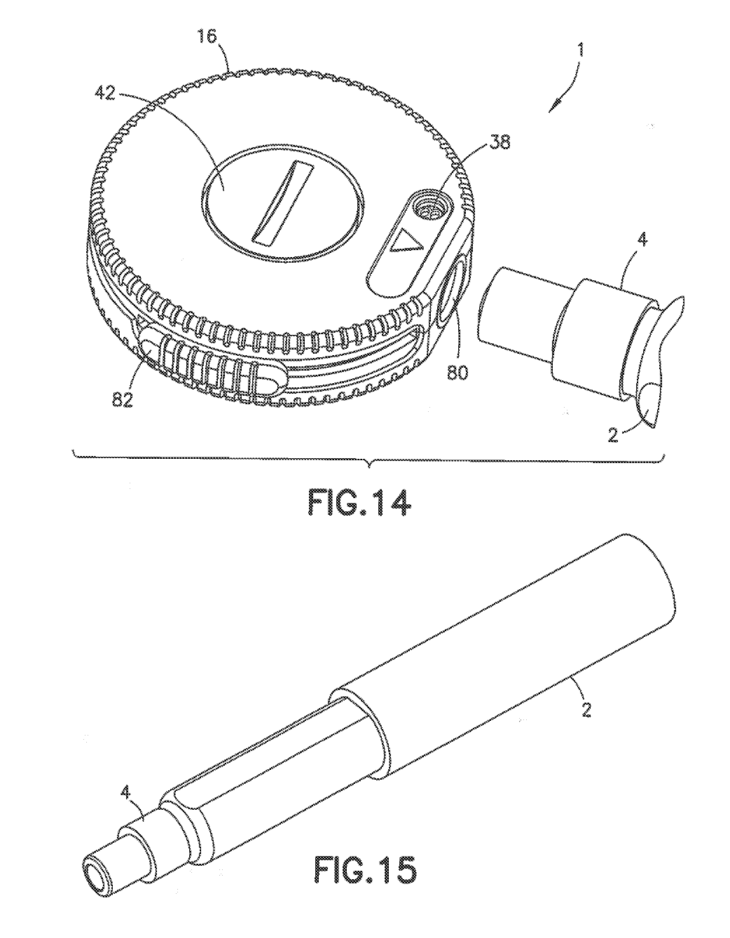

As a result, the magazine assembly 1 of FIG. 14 illustrates that the user can remove a needle hub 50 from the radial magazine 16 with a medication pen 2 via an adapter 4 for medication delivery. When the radial magazine 16 is indexed as each needle hub 50 is used, the hub chamber number 64 changes incrementally and is visible to the user through the window 38. Thus, the user can monitor the number of needle hubs 50 used and how many are remaining. Further details of the medication pen 2 and the adapter 4 are described below.

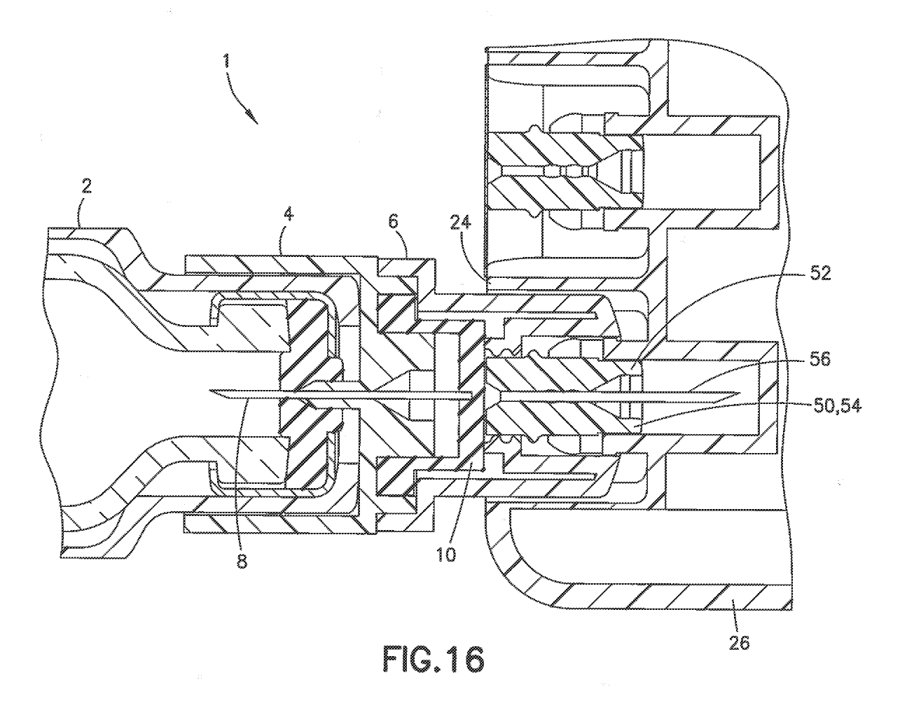

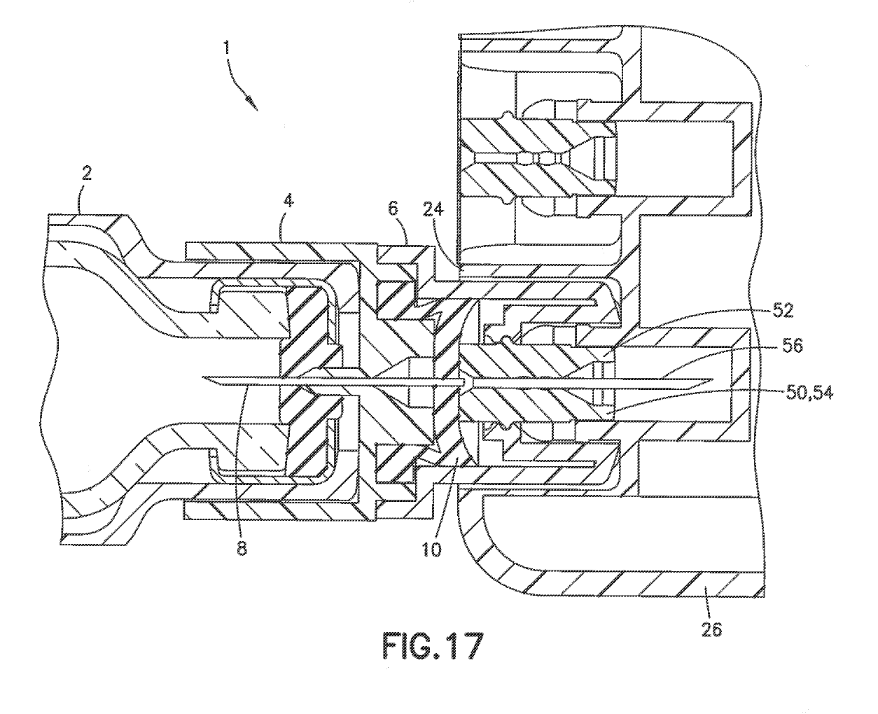

FIG. 15, according to one embodiment, illustrates the medication pen 2 connected to the adapter 4. The adapter 4 is attachable to a standard reusable medication pen 2 (i.e. pen injector). The components of the adapter 4 are illustrated in FIGS. 16 and 17. FIG. 16 illustrates the adapter 4 connected to the medication pen 2 and beginning to engage the selected needle hub 52. The adapter 4 includes an adapter body 6, an adapter cannula 8 and an adapter septum 10. The adapter body 6 is preferably a two piece press-fit assembly that encloses and secures the adapter septum 10. A proximal end of the adapter body 6 includes threads that are configured to engage threads on the medication pen 2. A distal end of the adapter body 6 includes an opening. The opening is configured to engage one of a plurality of needle hubs 50.

The adapter cannula 8 is fixed to the adapter body 6. The adapter cannula 8 is disposed in the proximal end of the adapter body 6 and is configured to pierce a septum of the medication pen 2 to establish fluid communication with the medication pen 2. A distal end of the adapter cannula 8 is disposed in the adapter septum 10.

Preferably, the adapter septum 10 includes a preformed opening for the adapter cannula 8 to pierce. Alternately, a sharpened distal end of the adapter cannula 8 pierces the adapter septum 10 to establish fluid communication. The adapter septum 10 is preferably made of silicon rubber. Operation of the adapter cannula 8 with the adapter septum 10 is described below.

The adapter septum 10 regulates the flow of medicament between the medication pen 2 and the selected needle hub 52. The adapter septum 10 is closed in its natural state. As illustrated in FIG. 16, the adapter septum 10 partially engages the adapter cannula 8 at its distal end in the closed position. Since the selected needle hub 52 in not fully engaged with the adapter 4, the adapter septum 10 remains in the closed position. That is, the adapter septum 10 of FIG. 16 is in its natural state.

FIG. 17 illustrates the adapter septum 10 in an open position. The selected needle hub 52 is fixed to the adapter body 6 via a push-pull detest although other methods are contemplated. When the selected needle hub 52 is fixed to the adapter body 6, the selected needle hub 52 applies an axial force to the adapter septum 10. The axial force causes the adapter septum 10 to flex (or compress) and allows the adapter cannula 8 to pierce the adapter septum 10 and extend into the selected needle hub 52. The axial force also establishes a sealing surface to prevent a leak path at an interface between a proximal end of the selected needle hub 52 and a distal surface of the adapter septum 10. Accordingly, the selected needle hub 52 is now in fluid communication with the medication pen 2 through the hollow adapter cannula 8.

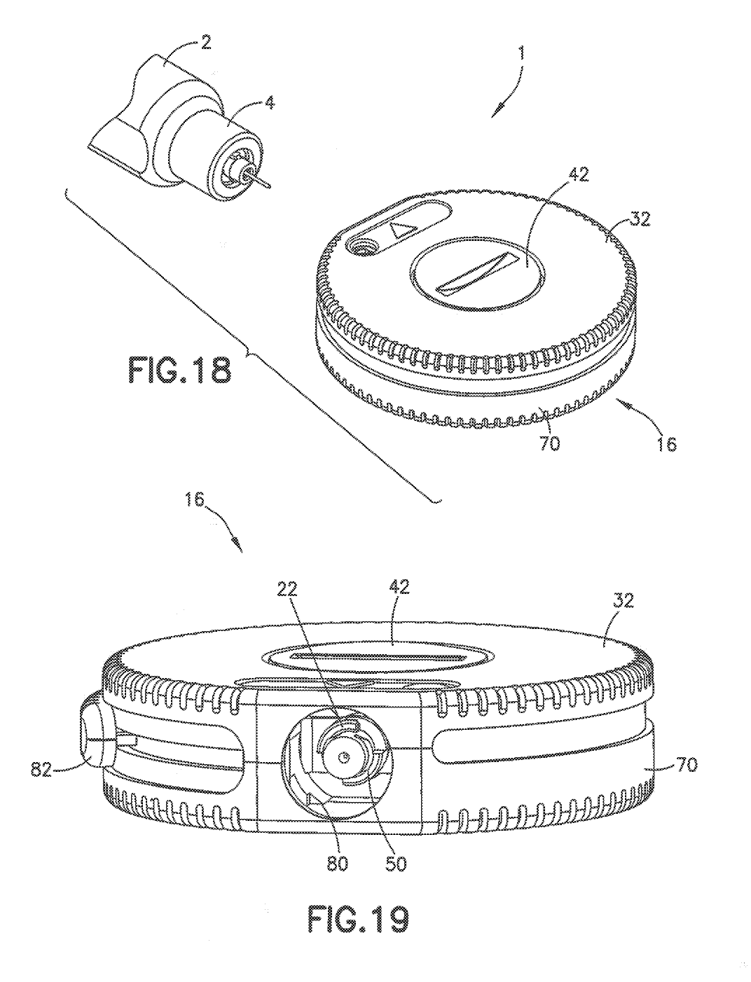

According to one embodiment, FIG. 18 illustrates the magazine assembly 1 where the selected needle hub 52 is connected to the medication pen 2 via the adapter 4 and is removed from the radial magazine 16. Specifically, the selected needle hub 52 disengages the connector 28 in the selected hub chamber 24 of the magazine array 26. Each of the plurality of needle hubs 50 cannot be easily removed from the radial magazine 16 without using the adapter 4. The medication pen 2 is now ready for medication delivery.

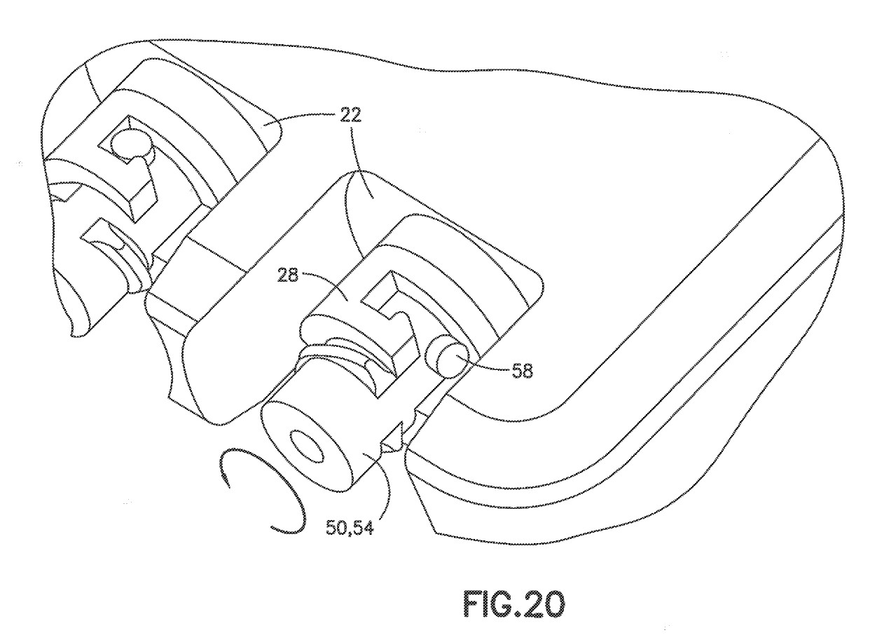

FIG. 19 illustrates, according to one embodiment, that after the selected needle hub 52 is used for medication delivery by the medication pen 2, the medication pen 2 returns the selected needle hub 52 back to the radial magazine 16. Specifically, the needle hub 50 engages the connector 28 in the hub chamber 22 of the magazine array 26. The needle hub 50 returns to the hub chamber 22 that it was originally sealed in. After use, the needle hub is no longer a selected needle hub.

Subsequently, the radial magazine 16 is indexed as described above and the process is repeated. After all the needle hubs 50 are used, the radial magazine 16 can no longer be indexed because the magazine rib 27 contacts the rib 36 in the top cover 32 to prevent further rotation. The radial magazine array 26 can be disassembled from the magazine housing 32, 70 and replaced. Thus, the radial magazine 16 can continue to store and provide pen needles for use in medication pens.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated, This description is not necessarily intended to be exhaustive or to limit the invention to the precise embodiments disclosed. Any of the embodiments and/or elements disclosed herein may be combined with one another to form various additional embodiments not specifically disclosed, as long as they do not contradict each other. Accordingly, additional embodiments are possible and are intended to be encompassed within this specification and the scope of the invention. The specification describes specific examples to accomplish a more general goal that may be accomplished in another way.

As used in this application, the terms “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately” are understood by those of ordinary skill to refer to reasonable ranges outside of the given value, for example, general tolerances associated with manufacturing, assembly, and use of the described embodiments.

1. A magazine that stores a plurality of needles configured to engage a medication pen for medication delivery, the magazine comprising:
a magazine housing enclosing
a removable array that is rotatable in the magazine housing, the removable array supporting a plurality of hub chambers each enclosing one of a plurality of needle hubs, the plurality of hub chambers being radially disposed;
a connector in each of the plurality of hub chambers, each connector engaging one of the plurality of needle hubs; and
a plurality of closures each sealing the plurality of needle hubs, wherein
a selected closure of the plurality of closures is pierced by the medication pen to engage a selected needle hub of the plurality of needle hubs, the selected needle hub disengages from the hub chamber, and the selected needle hub is removed from the magazine housing to prepare the medication pen for medication delivery.

2. The magazine of claim 1, wherein a centerline of each of the plurality of hub chambers substantially intersects at a centerline of the magazine housing.

3. The magazine of claim 1, wherein the magazine housing is a substantially circular shape.

4. The magazine of claim 1, wherein
the magazine housing includes a cover and a base, and
the magazine further includes a fastener to secure the cover to the base.

5. The magazine of claim 1, wherein the magazine housing includes a window to display a number identifying the needle hub in use.

6. The magazine of claim 1, wherein
the magazine housing includes an opening to provide access to the selected needle hub, and
the opening provides access to only one needle hub at a time.

7. The magazine of claim 6, wherein the opening is aligned to the selected needle hub.

8. The magazine of claim 1, further comprising a ratcheting slider for indexing the plurality of hub chambers in the magazine housing.

9. The magazine of claim 8, wherein the ratcheting slider includes a ratchet shield that obstructs an opening in the magazine housing during indexing.

10. The magazine of claim 8, wherein the ratchet shield provides access to the opening before and after indexing.

11. The magazine of claim 8, wherein the ratcheting slider moves along an outer circumference of the magazine housing.

12. The magazine of claim 1, wherein the magazine housing and the removable array each includes a rib that engages each other to prevent the magazine from exposing the plurality of needle hubs that are used.

13. The magazine of claim 1, wherein the removable array is replaced when the plurality of needle hubs is used.

14. The magazine of claim 1, wherein each of the hub chambers is identified by a hub chamber number.

15. The magazine of claim 14, wherein one of the hub chamber numbers aligns with a window in the magazine housing for display.

16. The magazine of claim 1, wherein
the magazine housing further includes a guide track to engage and disengage ratcheting slider.

17. The magazine of claim 16, wherein
the magazine housing further includes a ramp, and
the ramp is adjacent to the guide track to engage and disengage the ratcheting slider from the removable array.

18. The magazine of claim 1, wherein the magazine housing further includes a locking member to engage the removable array and prevent reverse rotation during indexing.

19. A method of using a plurality of needles in a magazine, the plurality of needles is configured to engage a medication pen for medication delivery, the method comprising:
indexing a selected needle hub of a plurality of needle hubs to align with an opening in a magazine housing;
piercing a selected closure of a plurality of closures that seals the selected needle hub with the medication pen;
engaging the medication pen to the selected needle hub in the magazine housing;
disengaging the selected needle hub from the magazine housing; and
removing the selected needle hub from the magazine housing to prepare the medication pen for medication delivery.
